(19) United States
(12) Patent Application Publication
(10) Pub. No.: US 2007/0184123 A1
(43) Pub. Date: Aug. 9, 2007

(54) Cosmetic Mixture For Hair

(76) Inventor: Atika Soulimani, Acquedolci (IT)

Correspondence Address: IP STRATEGIES, 12 1/2 WALL STREET, SUITE I, ASHEVILLE, NC, 28801, US

(21) Appl. No.: 11/578,210

(22) PCT Filed: Apr. 7, 2005
(86) PCT No.: PCT/IT05/00196

§ 371 Date: Oct. 5, 2006

(30) Foreign Application Priority Data
Apr. 7, 2004 (IT) RM2004A000177

(51) Int. Cl. A61K 36/8962; A61K 36/81; A61K 36/736; A61K 36/53; A61K 35/54

(52) U.S. Cl. 424/581; 424/751; 424/745; 424/754; 424/735

(57) ABSTRACT

The present invention is based on surprising discovery that a detailed selection and mixture of manufactures extracted from four different kinds of plants (hundreds of officinal plants avalaible) containing aviary egg deutoplasm (or egg yolk or calf) have permitted creating a new mixture with an efficacious no-dandruff effect together exactly as efficacious prevention effect for loss of hair or coat and stimulating the same growth again.

Therefore, term of the present invention is a mixture including manufactures of leaves as Nicotiana tabacum, from entire plant of Lavanda Officinalis, or bulb of Alliums pp., seed oil of Prunus Amygdalus dulcis and aviary egg deutoplasm, procedures for its manufacturing, cosmetic products including the above-mentioned mixture, procedures for its manufacturing and its utilization. Although these plants are well-known, it's unknown mixtures including a selection of manufactures of these plants in addition to aviary egg yolk. The union of these components, proved in the present invention, bringing to create a particular mixture well as no-dandruff, capable to stimulate a growth-again of the hair or coat and to prevent loss of them, devoid from secondary effects annoying for all users.

### TECHNICAL FIELD

The present invention consist of a cosmetic solution, suitable either for human or animal use, made up of natural components very helpful to take care of the scalp, to prevent dandruff arisen and to reduce the loss of hair or coat.

### BACKGROUND ART

Alopecia, partial or total, dandruff and the opacity both of the hair and the surface are two troubles that usually afflict both the men and animals.

Alopecia, that can depend either on hormonal or pathologic causes, consist of the partial or total shortage of hair, it's common knowledge that can bring a negative psychological effect on people who suffers and unaesthetic.

Generally the treatment consist of topical compositions, pills or systems that are all known procedures to involve secondary effects like stained growth, slow progression and irritation of the scalp or animal skin and diseases affecting the digestive system. Dandruff, instead, is caused from accelerated replacement of epidermic cells that, because of increase the speed of migration, don't be able to become completely mature before detaching itself. Take place white or greyish scales (heaps of corneous cells) localized in scattered stains and, more often, largely arranged on entire scalp or animal skin.

The itchiness is poor or absent. Because of the irregular disposition of the corneous cells in dandruff, in opposite of that commonly thinking, the corneous layer is more permeable than another with a normal skin and is necessary to remember it for avoiding unexpected systemic effects caused from active pharmacological substances applied locally with topical compositions.

At present many different products for to prevent loss of hair are known, these are able to stimulate a growth-again like that no-dandruff products effects are known.

Know as the weakness of the skin on subjects who suffers dandruff and who show loss of hair, not all famous products used to prevent the loss of hair (or coat), or able to stimulate a growth-again, are suitable for the latter, or rather, theirs utilization can be particularly unsuitable.

In the past was prepared some no-alopecia compositions including, as active principles, like pentadecanoato of gliceril, Swertia fluid extract, soluble cisteine, salicilic acid and others substances that are no good tolerating from scalp and animal skin.

For disadvantage, some of that compositions containing also active ingredients resulting from plant's extracts and are used too, like no-alopecia substances, plant's extracts according to officinal plant as Orchidacee, Melicacee, Liliacee, Poacee e Bombacanacee.

Even if some of them, like that according to Poacee and Bombacanacee seems don't bring irritation to scalp, doesn't mentioned, about the latter, no-dandruff effect together no-alopecia effect.

Therefore is hoped have new formulations that linking both characteristics (preventing loss and/or growth again and no-dandruff effect) in addition to a high tolerability from damaged skin because of dandruff presence.

Know as the actual tendency to utilize as much as possible natural products and don't use synthetic/chemical products, moreover it's recommendable that these formulations are based only natural products.

### DISCLOSURE OF INVENTION

The selections of plants are the followings:

Nicotiana tabacum, or tobacco, which leaves containing substances with emetic topic effect, no-scabies and insecticide;

Lavanda officinalis, or lavender, which plant containing substances with no-spasm effect, sedative, tonic and carminative;

Alliums pp., or garlic, which bulb containing substances able to extend artery, general antiseptic, anti-uric, diuretic and bactericide;

Prugnus amygdalus dulcis, or sweet almond tree, which pips containing a protective oil, softener and nourishing for skin.

The cosmetic mixture of the invention including manufacturers extracted from leaves of Nicotiana tabacum, from entire plant of Lavanda officinalis, from bulb of Alliums pp., from seed-oil of Prunus Amygdalus dulcis and aviary egg deutoplasm.

These manufactures can be, in case of Nicotiana tabacum leaves, from Lavanda officinalis entire plant, from Alliums pp. bulb, depend on the quality of used materials, fresh or dried, meal, fragment, or powder.

In case of Prunus amygdalus dulcis, this manufactures are in seed-oil shape.

The aviary eggs deutoplasm can belong to any kind of birds, obvious are recommendable eggs commonly in commerce like those about birds of Anseriformes order (ex. duck), Galliformes order (ex. hen, pheasant and so on), Columbiformes order (ex. pigeon, turtledove and so on). The egg yolk can be used fresh or freeze-dried shape, in case added with water. In the optimum form to realization, the Alliums pp. manufactures are Allium sativum (common garlic) bulb-grinded and can be prepared with fresh material, while the Nicotiana tabacum o Lavanda officinalis manufactures no-dependence can be prepared with fresh or dried material and is recommendable in meal or powder shape.

The cosmetic mixture of the invention including dried leaves manufactures of Nicotiana tabacum from 0.7 to 1.2% in weight, dried plant manufacture of Lavanda officinalis from 0.7 to 1.2%, Alliums pp. manufactures from 7 to 12% in weight, aviary egg deutoplasm from 32 to 42% in weight and seed-oil of Prunus amygdalus dulcis from 48 to 55% in weight.

In the favourite shape of manufacturing, it's including about the 0.9% in weight of Nicotiana tabacum dried leaves, about the 9% in weight of Allium spp. manufacture, about the 37% in weight of aviary egg deutoplasm and about the 52% in weight of Prunus amygdalus dulcis seed-oil.

The preparation of tobacco, lavender and garlic manufactures can be made through pestle or any other tool or device able to break into small pieces, to grind or pulverize the used vegetable materials.

Favourably, can be used too, as Nicotiana tabacum manufacture, the common sniff tobacco.

The oil of sweet almonds can be any seed-oil commonly in commerce and the egg yolk, favourable of hen, can be fresh or freeze-dried.

In case of utilization freeze-dried egg yolk, it can be directly introduced in the powder mixture or can be re-arranged with water before resulting about the 32-42% in weight of the mixture.

In the procedure of mixture preparation, Nicotiana tabacum manufactures, Lavanda officinalis manufactures, Alliums pp. bulb manufactures, Prunus amygdalus dulcis seed-oil, aviary egg deutoplasm are mixed in manner to create a homogeneous heap or, in case of become used the egg yolk in freeze-dried shape not arranged with water, a suspension.

The cosmetic product of this invention can be the same mixture or a mixture including excipients in cosmetic way acceptable, preserves, anti-oxidizers, bactericides, anti-mould, acids, colourings and/or perfumes.

In the cosmetic preparation, can be added to mixture of invention all those excipients, preserves, anti-oxidizers, bactericides, anti-mould, make acids, colourings and perfumes known to technician like suitable to be added in a cosmetic product.

The quantity of these substances can be changed, depend on kind of cosmetic product that we wish to realize, like a lotion, a cream, an emulsion, a suspension, a balsam or other and these can be added in definite quantity from technician.

The cosmetic product of this invention can be packaged on conservable way in suitable containers to contain material for more applications or in only-dose containers that can be loose maintained or in further way packaged in suitable containers capable to contain more only-dose shapes packages.

The cosmetic product of the invention is used through directly application on interested zone, i.e. scalp or animal skin. It's arranged in uniform way on tissue and leaved on pose-way for a period inclusive between 10 minutes and 5 hours.

For advantage, the product can be used during the presence of an heat-source like, for instance, hairdryer, helmet for put in fold, infrared ray lights, or others suitable heat-sources.

In this situation, the application can keep for a period inclusive between 10 and 30 minutes, it's recommendable at least 15 minutes. The heat-sources can be plan out to generate a higher temperature from 37° C. and a lower temperature from 70° C., for instance, when utilizing an hairdryer, the optimum temperature of the air-jet is about 57° C. Instead when there isn't a suitable heat-source, that is the case which the product equal the body-temperature, it's recommendable to maintain the application for a period inclusive between 2 and 5 hours, in favourably way at least 3 hours.

The following instances are just explanatory and don't want to limit the purpose of invention.

### EXAMPLES

### Ex. 1

The preparation for only-dose mixture:

Later on finely grinded, for to reduce in powder shape, 0.5 gram of Lavanda officinalis plant and finely grinded 5 gram of fresh common garlic, excluding protective layer.

These manufactures are been mixed together, and added 0.5 gram of sniff-tobacco in powder shape, a hen fresh egg yolk and 28 millilitres of sweet almond-oil. All together mixed to obtain a homogeneous heap.

### Ex. 2

The mixture use according to example 1 like cosmetic:

It was applied, in uniform way, the mixture prepared according to example 1 on the scalp about one hundred of people that show dandruff and/or evident loss of hair. This application is carried out, like on further description, more times during about 2 months.

The mixture is leaved every time for a period about 20 minutes and warm up through an hairdryer. Later on the mixture is eliminated with plentiful rinsing and the hair washed with a common shampoo.

The application of the mixture like description has proved in all cases to dandruff disappearance, to stop loss of hair and to growth-again of the hair on dispersed areas.

1. Cosmetic mixture characterized by a detailed manufactures selection and mixture of Nicotiana tabacum leaves, of entire plant of Lavanda Officinalis, of bulb of Alliums spp. and seed oil of Prunus Amygdalus dulcis, that, mixed with aviary egg deutoplasm (or egg yolk or calf) has permitted creating a new mixture with amazing efficacious no-alopecia and no-dandruff effects together exactly as efficacious prevention effect for loss of hair or coat and stimulating the same growth again, devoid from secondary effects annoying for all users.

2. Mixture, as claimed in claim 1, including the deutoplasm that is extracted from bird's eggs of Anseriformes order, Galli-formes order, Columbiformes order.

3. Mixture, as claimed in claim 1, including Nicotiana tabacum and Lavanda officinalis manufactures, obtained from dried materials and show in small pieces, grinded or powder way and the Allium spp. manufactures are obtained from fresh material in grinded or fragmentary shape.

4. Mixture, as claimed in claim 1, including the Nicotiana tabacum dried leaves manufacture from 0.7 to 1.2% in weight, the Lavanda officinalis dried plant manufacture from 0.7 to 1.2% in weight, the Allium spp. manufacture from 7 to 12% in weight, the aviary egg deutoplasm from 32 to 42% and the Prunus amygdalus dulcis seed-oil manufacture from 48 to 55%.

5. Mixture, as claimed in claim 1, including the Nicotiana tabacum dried leaves manufacture from 0.9% in weight, the Lavanda officinalis dried plant manufacture about 0.9% in weight, the Allium spp. manufacture about 9% in weight, the aviary egg deutoplasm about 37% in weight and the Prunus amygdalus dulcis seed-oil about 52% in weight.

6. Cosmetic mixture, as claimed in claim 1, characterized from the fact that is suitable either for human or animal use.

7. Cosmetic mixture, as claimed in claim 1, characterized from the fact to have amazing efficacious no-alopecia and no-dandruff effects together exactly as efficacious prevention effect for loss of hair or coat and stimulating the same growth again, devoid from secondary effects annoying for all users.

8. Cosmetic mixture, as claimed in claim 1, characterized from the fact to have high tolerability from damaged skin.

9. Cosmetic product including mixture, as claimed in claim 1, optionally cosmetic acceptable excipients, preserves, anti-oxidizers, bactericide, anti-mould, acids, colourings and/or perfumes.

10. Procedure for mixture preparation or cosmetic product, as claimed in claim 1, in which Nicotiana tabacum leaves manufacture, Lavanda officinalis entire plant manufacture, Allium spp. bulb manufacture, Prunus amygdalus dulcis seed-oil manufacture, and aviary egg deutoplasm are mixed all together in any order to obtain an homogeneous mass.

11. Procedure, as claimed in claim 1, including the mixture with cosmetic acceptable excipients, preserves, anti-oxidizers, colouring and/or perfumes in conservable form.

12. The utilization of the cosmetic product, as claimed in claim 1, in which this cosmetic applied in uniform way on the scalp or animal skin and is maintained for a period between 10 minutes and 5 hours, exceptionally warmed-up on higher temperature of 37° C. and lower temperature of 70° C.

13. Utilization, as claimed in claim 1, this cosmetic is maintained for a period between 10 minutes and 30 minutes warmed-up on higher temperature of 37° C. and lower temperature of 70° C.

14. Utilization, as claimed in claim 1, this cosmetic is maintained for a period between 2 hours and 5 hours on body temperature.
